(19) United States
(12) Patent Application Publication
(10) Pub. No.: US 2018/0271704 A1
(43) Pub. Date: Sep. 27, 2018

(54) OPHTHALMIC SURGICAL DEVICE FOR CUTTING A CIRCULAR INCISION

(71) Applicant: MOR RESEARCH APPLICATIONS LTD., Tel-Aviv (IL)

(72) Inventors: Tova LIFSHITZ, Lehavim (IL); Nadav Yaakov BELFAIR, Beer Sheva (IL); Eliyahu BSHARIM, Beer Sheva (IL); Eliahu ELIACHAR, Haifa (IL); Nir LILACH, Carmiel (IL); Nathan BUKHDRUKER, Carmiel (IL)

(21) Appl. No.: 15/571,289

(22) PCT Filed: May 3, 2016
(86) PCT No.: PCT/IL2016/050456

§ 371 Date: Nov. 2, 2017

Related U.S. Application Data
(60) Provisional application No. 62/156,272, filed on May 3, 2015.
(60) Provisional application No. 62/162,821, filed on May 18, 2015.

(51) Int. Cl. A61F 9/007

(52) U.S. Cl. CPC A61F 9/00754; A61F 9/00763

(57) ABSTRACT

Aspects of embodiments relate to a surgical device for assisting a user of the device to cut a circular incision in an anterior lens capsule tissue, the device comprising an arm member having a proximal end and a distal end, wherein the distal end of the arm member is operative to receive a cutting member; and an actuator arrangement comprising an actuating handle operatively coupled with the arm member, such that, responsive to an operating input provided at the actuating handle, the distal end of the arm member traverses a circular path around a first axis of the tissue to cause the cutting member to form a circular incision in the tissue along the circular path.

### TECHNICAL FIELD

Embodiments disclosed herein relate in general to surgical devices and methods for performing microsurgery of tissue.

### BACKGROUND

Among other factors, the quality of vision depends upon the transparency of an individual's lens. The lens, which focuses light entering the eye onto the retina of the eye, is supposed to be transparent to allow for unobstructed vision. Hence, opacity or cloudiness of the lens may prevent a clear image from forming on the retina, resulting in impairment or loss of vision. This condition is commonly known as a cataract, which is a leading cause of blindness worldwide.

If the lens develops cloudy or opaque areas, the lens must be surgically removed. To date, surgical treatment by cataract removal is the preferred treatment, in which the lens is be replaced with an artificial intraocular lens (IOL) to provide better vision after cataract removal.

The lens to be replaced is encapsulated by a cellophane-like membrane tissue covering the anterior and posterior surfaces of the lens forming the lens capsule. The surgical treatment includes the procedure of creating an opening in the membrane tissue of desired diameter and shape. Tears or defects on the edge of the opening make the lens capsule comparably weak and, therefore, vulnerable to losing the ability to properly hold the IOL, along with reduced stability during phacoemulsification for emulsifying the lens nucleus.

Creating an opening of desired diameter and shape, i.e., without tears or defects for example, and which is centered on the optical axis, is referred to as curvilinear continuous capsulorhexis (CCC).

Creating such opening in the lens capsule with the required level of precision to achieve CCC is a relatively challenging task.

The description above is presented as a general overview of related art in this field and should not be construed as an admission that any of the information it contains constitutes prior art against the present patent application.

### SUMMARY

Aspects of disclosed embodiments relate to a surgical device for assisting a user in cutting a circular incision in a tissue, wherein the tissue is an anterior lens capsule tissue. It is noted that the term “circular” also encompasses the meaning of the term “substantially circular” and may refer to any (open or closed) annular path. Correspondingly, the term “circular” does not necessarily have to refer to a perfect circular shape, but may also encompass an about circular, about elliptical, or other closed loop shape.

Example 1 comprises a surgical device that includes an arm member having a proximal end and a distal end, wherein the distal end of the arm member is operative to receive a cutting member; and an actuator arrangement comprising an actuating handle operatively coupled with the arm member such that, responsive to an operating input provided at the actuating handle, the distal end of the arm member traverses a circular path around a first axis of the tissue to cause the cutting member to form a circular incision in the tissue along the circular path.

Example 2 includes the subject matter of example 1 and, optionally, wherein the actuator arrangement is manually operable with one-hand through the actuating handle.

Example 3 includes the subject matter of examples 1 or 2 and, optionally, further comprises a tissue-engaging support member extending through a tube-shaped portion of the actuator arrangement, for providing support to the surgical device and fixating the position of the surgical device relative to the tissue.

Example 4 includes the subject matter of example 3 and, optionally, wherein the tissue-engaging support member is rotatable.

Example 5 includes the subject matter of example 4 and, optionally, wherein the tissue-engaging support member comprises form-locking engagement elements.

Example 6 includes the subject matter of any of the preceding examples and, optionally, wherein the rotatable arm member is operative to receive a cutting member having a circular blade rotatable around a second axis which is about perpendicular to a first rotational axis of the arm member.

Example 7 includes the subject matter of any of the preceding examples and, optionally, wherein the rotatable arm member is operative to receive an L-shaped cutting member.

Example 8 includes the subject matter of any of the preceding example and, optionally, further comprises a handle for allowing the user to hold the device in one hand to allow one-handed operation of the device.

Example 9 includes the subject matter of any of the preceding example and, optionally, further comprises a tissue-gripper for gripping and lifting at least a portion of the tissue.

Example 10 comprises a method for cutting a circular incision in a tissue, where the tissue is an anterior lens capsule tissue. The method comprises the procedure of providing a surgical device comprising an arm member comprising a proximal end and a distal end, where the distal end of the arm member is operative to receive a cutting member; and an actuator arrangement comprising an actuating handle operatively coupled with the arm member. The method further comprises the procedure of applying an operating input at the actuating handle, such that the distal end of the arm member traverses a circular path around a first axis of the tissue to cause the cutting member to form a circular incision in the tissue along the circular path.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE FIGURES

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements. The figures are listed below.

FIGS. 1A to 1D are schematic three-dimensional view representations of a surgical instrument in respective sequential operative positions showing the progress of cutting a circular incision in the membrane tissue of a lens capsule with a cutting member, according to some embodiments;

FIG. 2 is an enlarged schematic three-dimensional view representation of the embodiment shown in FIG. 1A;

FIG. 3 is a partial side view illustration of the surgical instrument operably engaging the lens capsule, according to the embodiment of FIG. 2;

FIG. 4 is a schematic three-dimensional view representation of a surgical device with another cutting member, according to some embodiments;

FIGS. 5A, 5B and 5C are schematic side view illustrations of embodiments of tissue-engaging support members of the surgical device, according to some embodiments;

FIG. 6 is a partial side view illustration of the surgical instrument operably engaging the lens capsule, according to the embodiment of FIG. 4;

FIGS. 7A and 7B are schematic side view illustrations of a cutting member in extended and collapsed configuration, respectively;

FIGS. 8A and 8B are schematic side view illustrations of another cutting member in extended and collapsed configuration, respectively;

FIG. 9 is a schematic three-dimensional view representation of a surgical device, according to some other embodiment;

FIG. 10 is a partial side view illustration of the surgical instrument operably engaging the lens capsule, according to the embodiment of FIG. 9;

FIGS. 11A to 11D are schematic three-dimensional view representations of a surgical instrument in respective sequential operative positions showing the progress of cutting the membrane tissue of a lens capsule with a cutting member, according to the embodiments of FIGS. 9 and 10; and

FIGS. 12A and 12B are schematic three-dimensional view representations of a surgical instrument, according to some embodiments.

### DETAILED DESCRIPTION

The following description of devices and methods for creating precise openings in tissue of a desired diameter and shape are given with reference to particular examples, with the understanding that such devices and methods are not limited to these examples.

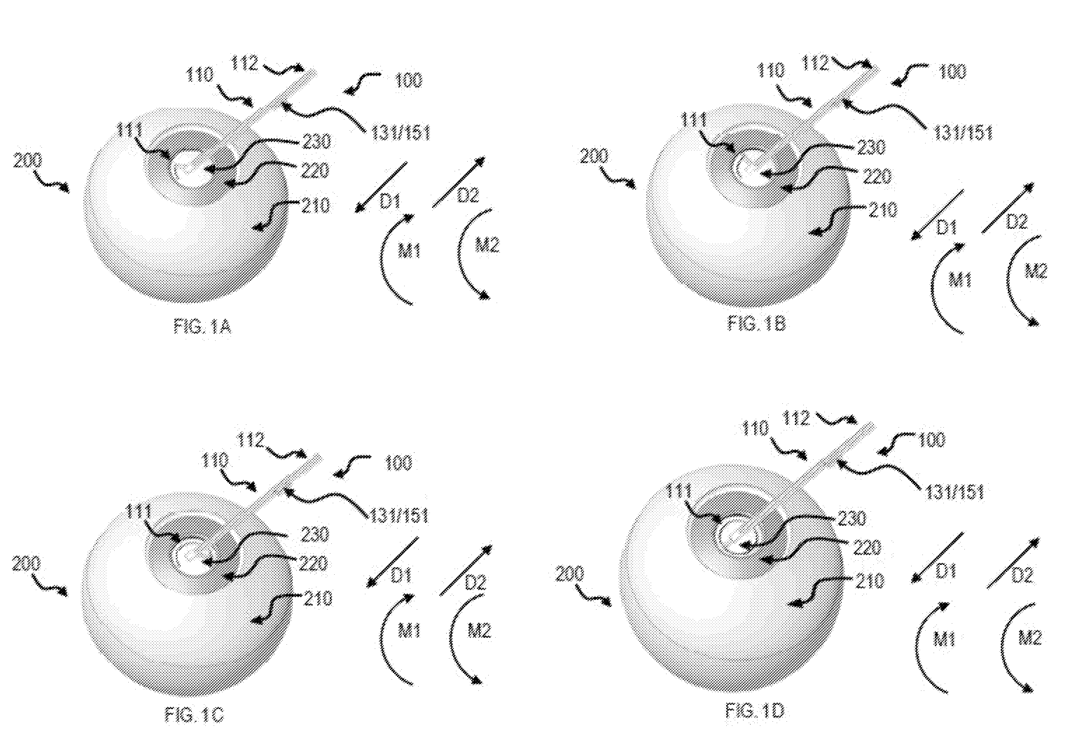

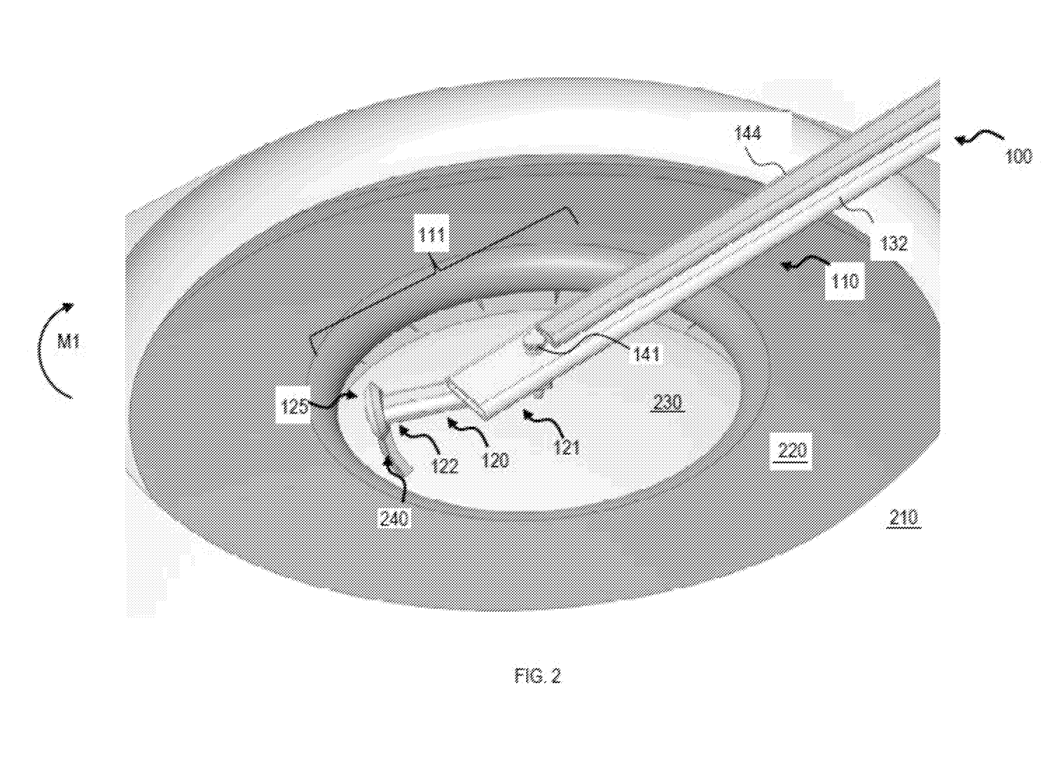

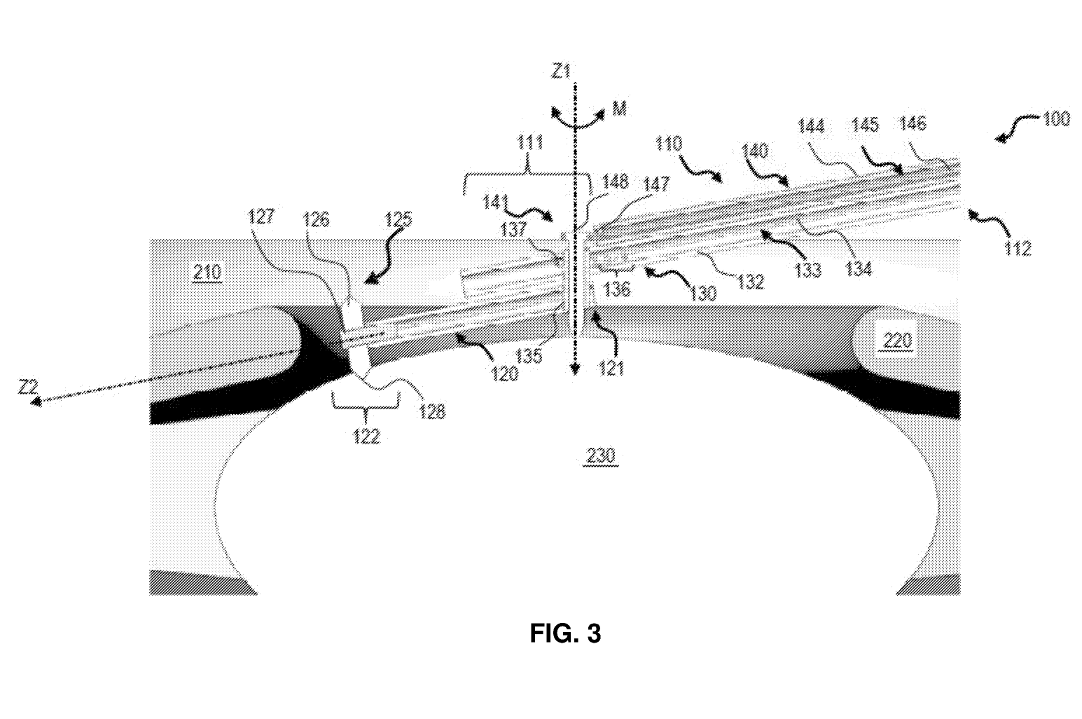

Reference is made to FIGS. 1A to 1D, and to FIGS. 2 and 3. A surgical device 100 suitable for performing a surgical procedure on, for example, an individual's eye 200, is described in the following. Surgical device 100 may thus, in some embodiments, also be referred to as an “ophthalmic surgical device”.

Accordingly, while embodiments of device 100 are disclosed herein with respect to ophthalmic surgical procedures, this should by no means to be construed as limiting.

The scleral tissue of the individual's eye 200 is herein designated by alphanumeric reference “210”, the iris by alphanumeric reference “220”, and the lens capsule along with its encapsulating membrane tissue by alphanumeric reference “230”.

Surgical device 100 comprises a handle member 110; an arm member 120 which may be operative to detachably receive a cutting member 125; an actuator arrangement 130, and a support member arrangement 140. In some embodiments, actuator arrangement and/or support member arrangement 140 may be coupled with, included in or constitute handle member 110. Actuator arrangement 130 is operatively coupled with arm member 120 such that by engaging actuator arrangement 130 the arm member is pivoted or rotated. In other words, arm member 120 is rotatably coupled with actuation arrangement 130. Support member arrangement 140 may be employed for fixating the position of handle member 110 relative to the tissue to be cut, as outlined herein below in greater detail. In an embodiment, the dimensions of handle member 110 may be such to be handy. Length of handle member 110 may for example range from about 10-15 cm.

In some embodiments, length of handle member 110 may be telescopically adjustable to easily fit hands of various sizes.

According to some embodiments, handle member 110 may be of longitudinal extension and have a distal handle portion 111 and a proximal handle portion 112. According to some embodiments, arm member 120 may be of longitudinal extension and have a proximal arm end 121 and a distal arm end 122, the distal arm end 122 operative to receive cutting member 125.

Arm member 120 may be rotatably coupled with actuator arrangement 130 such that, in response to operatively engaging actuator arrangement 130, arm member 120 and cutting member 125 coupled thereto rotate around a first rotation axis Z1. Surgical device 100 may be configured so that cutting member 125 may traverse along a substantially circular route R for cutting an about circular incision 240 in the membrane tissue encapsulating lens capsule 230. Cutting member 125 may for example traverse along a substantially circular route over a distance that corresponds to an angle of 360 degrees or less. Accordingly, as is schematically illustrated in FIG. 2 for example, when surgical device 100 operably engages a tissue, operating actuator arrangement 130 may cause cutting member 125 to cut incision 240 into the tissue, which may for example be a membrane tissue encapsulating lens capsule 230.

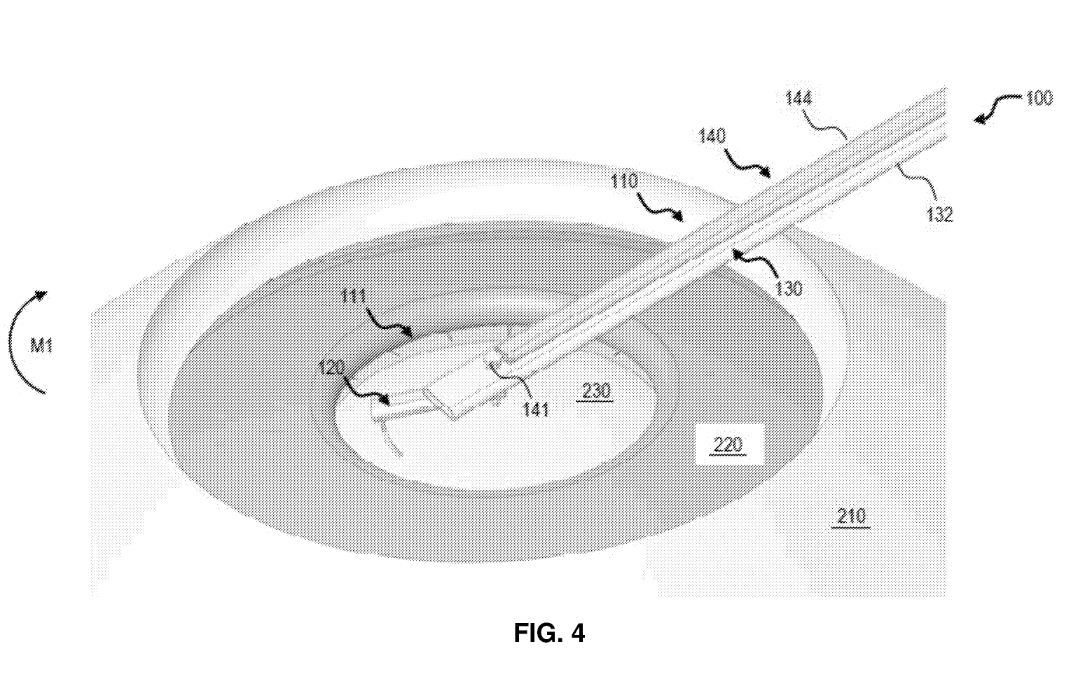

Additional reference is made to FIG. 4. Actuator arrangement 130 may for example comprise a system operative to translate, for example, a suitable operating or actuating input provided by a user of device 100 to a cutting-actuating handle 131 (implemented e.g., as a slidable element) into rotational movement of arm member 120 to cause cutting member 125 to traverse along a circular route for cutting an opening into lens capsule 230. Cutting member 125 may traverse to facilitate obtaining curvilinear continuous capsulorhexis (CCC).

In an embodiment, surgical device 100 may be configured so the radius of the circular route traversed by cutting member 125 is adjustable. In an embodiment, surgical device 100 may be configured so that movement of actuating handle 131 may be limited between two actuating positions and movement of actuating handle 131 starting from the first position and terminating at the second position of the two actuating positions causes cutting member 125 to traverse along a circular route to obtain CCC or, otherwise stated, a continuous, stress-free, tag-free and properly positioned circular opening in the anterior surface of lens capsule 230. In some embodiments, surgical device 100 may be configured to allow cutting member 125 to traverse an angle of 360 degrees at least. In some embodiments, surgical device 100 may be configured to allow cutting member to traverse an angle of less than 360 degrees. It is noted that the rotational angle may in some embodiments be adjustable by the user of device 100.

Such system may for example be implemented through pneumatic, hydraulic, mechanical gears and/or any other suitable cutting member rotating mechanism which can be engaged, e.g., one-handed operation by a user (not shown) of device 100. Such actuator arrangement 130 may be engagable while being free of an internal and/or external power source while allowing one-handed user operation of the device. The only source of power may be a mechanical input force provided by the user.

According to some embodiments, such cutting member rotating mechanism may be implemented in a manner as outlined in the following. The cutting member rotating mechanism may for example comprise a gear assembly which may for instance comprise an at least partially tube-shaped body 132 defining internal to the body a cutting-steering passageway 133 for at least partially housing a bendable cutting-steering rod 134 extending along the cutting-steering passageway. Cutting-steering rod 134 may frictionally and/or otherwise operatively engage with a shaft 135 such that a linear displacement of cutting-steering rod 134 causes rotation of shaft 135 and, therefore, of cutting member 125 attached thereto. Cutting-steering rod 134 may for example be held in position and guided by first cutting guide elements 136 within steering-cutting passageway, and frictionally wrapped by second circular cutting rod guide elements 137 around shaft 135. In some embodiments, circular cutting rod guide element 137 may be embodied by a pulley.

It is noted that the terms “clockwise direction M1” and “counterclockwise direction M2” discussed herein as well as grammatical variations thereof, refer to the rotational directions when viewing eye 200 in the propagation direction of light entering lens capsule 230.

Cutting-steering rod 134 may be linearly displaceable in a first distal direction D1 and a second, reverse or proximal direction D2, through selectively providing a first and a second input (e.g., manually) using cutting-actuating handle 131 (FIGS. 1A to 1D) coupled with rod 134 in a first and second operational manner. Actuating or engaging cutting-actuating handle 131 in the first operational manner, may result in the linear displacement of cutting-steering rod 134 towards distal handle portion 111. In turn, cutting-steering rod 134 may turn shaft 135 in a first rotational direction. Conversely, actuating or engaging cutting-actuating handle 131 in the second operational manner, may result in the linear displacement of cutting-steering rod 134 towards proximal handle portion 112. In turn, cutting-steering rod 134 may turn shaft 135 in the opposite direction and, thus, rotatable arm member 120 in a second rotational direction.

Depending for example on how shaft 135 and cutting-actuating handle 131 are coupled with cutting-steering rod 134, linear displacement of cutting-steering rod 134 in a distal direction D1 towards distal handle portion 111, may cause clockwise rotation of arm member 120. Accordingly, linear displacement of cutting-steering rod 134 in a proximal direction D2 towards proximal handle portion 112, may conversely cause counterclockwise rotation of arm member 120.

In some embodiments, gear mechanism may be configured so that linear displacement of cutting-steering rod 134 in a distal direction D1 towards distal handle portion 111, may cause counterclockwise rotation of arm member 120. Accordingly, linear displacement of cutting-steering rod 134 in a proximal direction D2 towards proximal handle portion 112, may conversely cause clockwise rotation of arm member 120.

In some embodiments, gear mechanism may be configured so that linear displacement of cutting-steering rod in distal direction D1 may be effected by sliding a slider member, which may embody cutting-actuating handle 131, in the same, distal, direction D1. Accordingly, linear displacement of cutting-steering rod in proximal direction D2 may be effected by sliding the slider member embodying cutting-actuating handle 131 in the same proximal direction D2.

In some other embodiments, gear mechanism may be configured so that linear displacement of cutting-steering rod in distal direction D1 may be effected by sliding a slider member embodying cutting-actuating handle 131 in the opposite, proximal, direction D2. Accordingly, linear displacement of cutting-steering rod in proximal direction D2 may be effected by sliding a slider member embodying cutting-actuating handle 131 in the opposite, distal direction D1.

Without being construed as limiting and merely to simplify the discussion that follows, displacement of cutting steering rod 134 in distal direction D1 (i.e., towards distal handle portion 111) is in the following considered to cause clockwise rotation, which is schematically indicated herein by arrow M1.

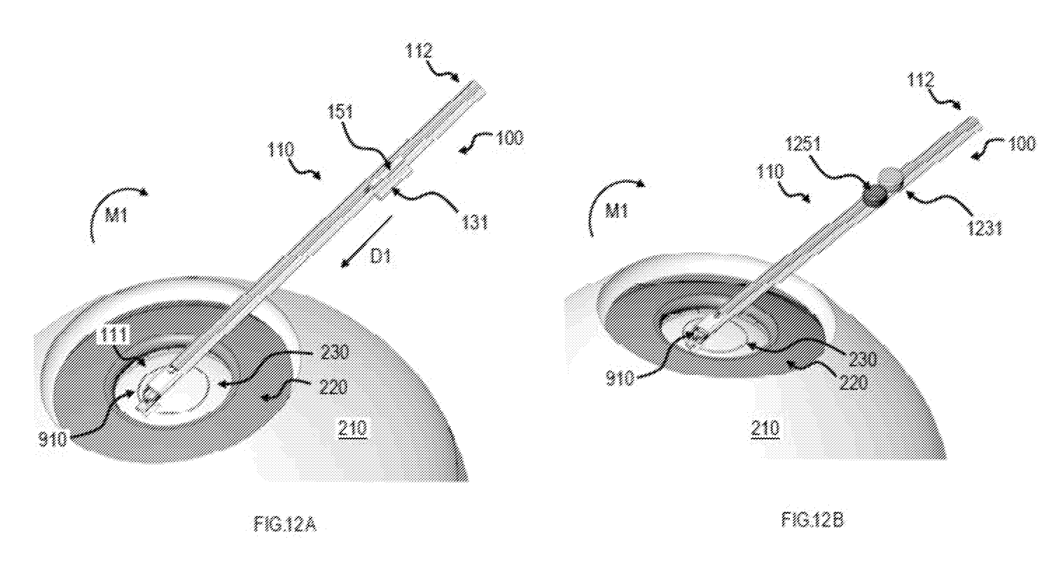

Clearly, a cutting-actuating handle may in some embodiments be implemented differently. A cutting-actuating handle may for instance be implemented as rotating knob 1231, as schematically shown in FIG. 12B. Depending on the direction of rotation of such knob 1231, cutting-steering rod 134 may be displaced in distal direction D1 or proximal direction D2, for respectively rotating cutting member 125 in a clockwise or a counterclockwise direction for example. As already outlined herein, displacement of cutting-steering rod 134 in distal direction D1 or proximal direction D2 may in some embodiments cause cutting member 125 to respectively rotate in a counterclockwise or a clockwise direction.

According to some embodiments, arm member 120 may only be rotatable in one direction only (e.g., in clockwise direction only), regardless of the input provided by the user at the actuating handle.

According to some embodiments, rotational velocity of arm member 120 is remotely controllable, e.g., manually, by the user of device 100, depending on the operational input provided at the actuating handle by the user.

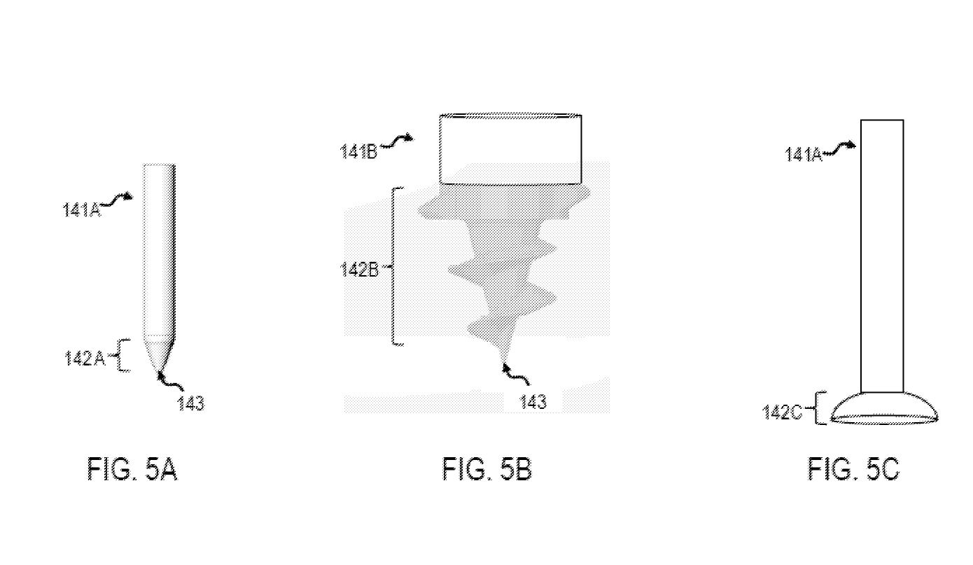

Reverting to FIGS. 1A to 1D and FIGS. 2 to 4, and further referring to FIGS. 5A-5C, support-member arrangement 140 of surgical device 100 may include a tissue-engaging support member 141 having an upper and lower end and which extends at distal handle portion 111 through the tube-shaped body 132 of actuator arrangement 130 as well as through proximal arm end 121 of arm member 120. In an embodiment, tissue-engaging support member 141 may be detachably mounted.

In some embodiments, tissue engaging support member 141 may extend through shaft 135 so that the longitudinal axis of tissue engaging support member 141 coincides with the rotational axis of the shaft. Accordingly, shaft 135 may have an elongate tube-shaped body with a hollow cavity for rotatably receiving, at least partially, tissue engaging support member 141.

According to some embodiments, tissue engaging support member 141 may provide support to surgical device 100 and fixate its position relative to the tissue to be cut. In some embodiments, tissue engaging support member 141 and shaft 135 may be identical.

Tissue engaging support member 141 may for example be positioned relative to cutting member 125 so that cutting member 125 can engage with a tissue for cutting an about circular opening therein while, at the same time, the lower end of tissue-engaging support member 141 can engage with a support surface. For example, when in an operable position for performing capsulorhexis, tissue engaging support member 141 may be at a position which may about coincide with the eye's optical axis Z1. Due to the convex form of lens capsule 230, the area where cutting member 125 engages lens capsule 230 is located below the engagement area of the lower end of tissue engaging support member 141 with the lens capsule, when viewed from the direction of light entering eye 200, i.e., from the top.

As shown schematically in FIGS. 5A-5B, tissue engaging support member 141 may in some embodiments have an elongate body which may, at least partially, taper towards the lower end thereof. For example, elongate body may have a tapering portion 142 which may, for instance, be cone-shaped, pyramid-shaped, and/or otherwise shaped. In some embodiments, lower tip portion may terminate in a tip 143, which may be about spherical, frustrum or otherwise shaped. A tapering portion may herein interchangeably be referred to as “tip portion”.

Tissue-engaging support member 141 may in one embodiment be free of form-locking engagement elements (FIG. 5A), whereas in another embodiment, tissue-engaging support member 141 may include form-locking engagement elements (FIG. 5B). For example, as shown schematically in FIG. 5B, such form-locking engagement elements may be embodied by screw-threads to facilitate the form-locking coupling of tip portion 1426 with lens capsule 230 (FIG. 5B). For example, as a result of the rotating of tissue-engaging support member 141, e.g., in a clockwise direction, tip portion 1426 may be screwed into lens capsule 230.

Referring to FIG. 5C, tissue-engaging support member 141 may have a suction cup 142C at its lower end for allowing vacuum-based fixation of the tissue engaging support member 141 to the tissue to be cut. Suction cup 142C may be made of a flexible and air-tight material and may have an about spherical shape having an outer and inner surface. The inner surface may have a concave shape facing, when in operable position, the surface to be cut.

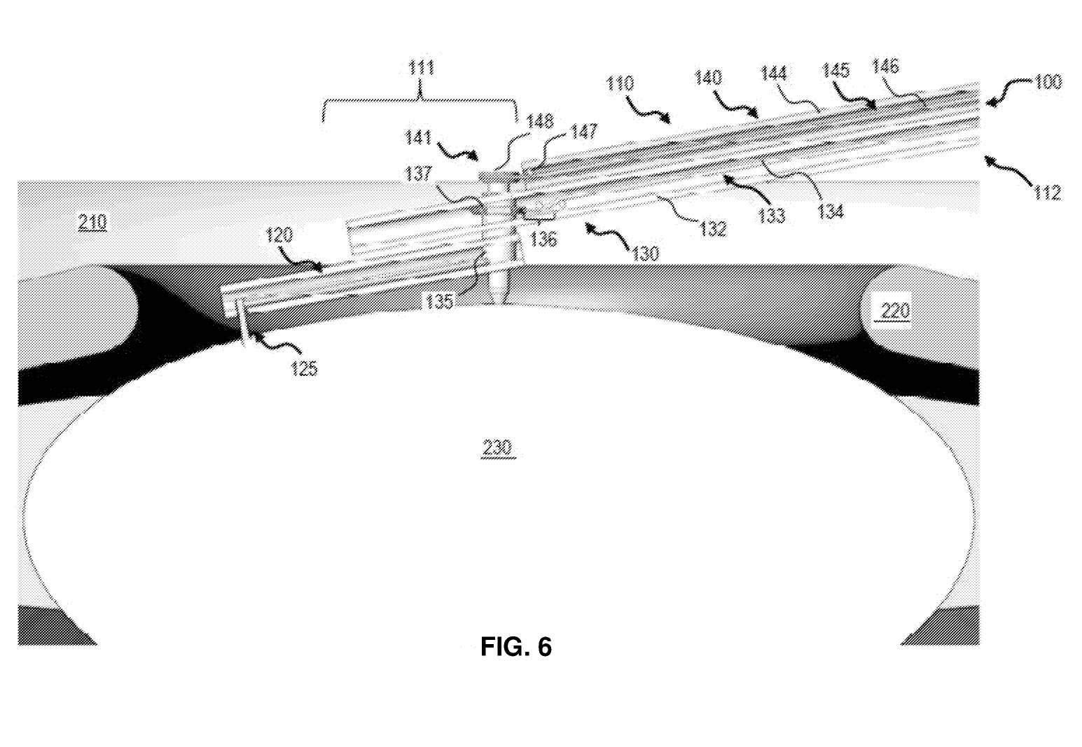

Additional reference is made to FIG. 6. Support member arrangement 140 may include a tissue engaging support member rotating mechanism, which may be implemented, in some embodiments, as outlined herein below. The support member rotating mechanism may, for example, be employed to rotate tissue engaging support member 141 having tip portion 142B comprising screw threads, as schematically shown in FIG. 5B.

Depending on the operating input provided by the user of device 100, the support member rotating mechanism may cause tissue engaging support member 141B to rotate either in a clockwise or a counterclockwise direction.

Support member arrangement 140 may for example comprise a system operative to translate, for example, a suitable operating or actuating input provided by a user of device 100 to a support member actuating handle (e.g., implemented as a slidable element 151) into rotational movement of arm member 120 to cause tissue-engaging support member 141 to rotate. Such a system may for example be implemented through pneumatic, hydraulic, mechanical gears and/or any other suitable cutting member rotating mechanism which can be engaged, e.g., through one-handed operation by a user (not shown) of device 100. Such support member arrangement 140 may be engagable while being free of an internal and/or external power source while allowing one-handed operation of device 100. The only source of power may be a mechanical input force provided by the user.

According to some embodiments, the support member rotating mechanism may for example comprise a support member gear assembly comprising an at least partially tube-shaped body 144 encompassing a support member steering passageway 145 for at least partially housing a bendable support member steering rod 146 extending along the support member steering passageway. Support member steering rod 146 may frictionally and/or otherwise operatively engage with tissue-engaging support member 141 such that a linear displacement of cutting-steering rod 134 causes rotation of the latter around rotational axis Z1.

According to some embodiments, support member steering rod 146 may be held in position and guided, for example, by first support member steering rod guide elements 147 within support member steering passageway 145 and further wrapped around a circular support member rod guide element 148, which may for example be a pulley.

Support member-steering rod 146 may be linearly displaceable in a first, distal, direction D1 and a second, reverse or proximal, direction D2, through selectively providing a first and a second input (e.g., manually) using support member actuating handle 151 (FIGS. 1A to 1D) coupled with rod 146, in a first and second operational manner. Actuating or engaging support member actuating handle 151 in the first operational manner, may result in the linear displacement of support member steering rod 146 towards proximal handle portion 112. In turn, support member steering rod 146 may rotate tissue-engaging support member 141 in a first rotational direction. Conversely, actuating or engaging support-member actuating handle 151 in the second operational manner, may result in the linear displacement of support-member steering rod 146 towards distal handle portion 111. In turn, support-member steering rod 146 may turn tissue-engaging support member 141 in a second rotational direction.

Depending for example on how tissue-engaging support member 141 and support-member actuating handle 151 are coupled with support-member steering rod 146, linear displacement of support-member steering rod 146 in distal direction D1 towards distal handle portion 111, may cause clockwise rotation of tissue-engaging support member 141. Accordingly, linear displacement of support-member steering rod 146 in a proximal direction D2 towards proximal handle portion 112, may conversely cause counterclockwise rotation of tissue-engaging support member 141.

In some other embodiments, the support-member rotating gear mechanism may be configured so that linear displacement of support-member steering rod 146 in a distal direction D1 towards distal handle portion 111, may cause counterclockwise rotation of tissue-engaging support member 141. Accordingly, linear displacement of support-member steering rod 146 in a proximal direction D2 towards proximal handle portion 112, may conversely cause clockwise rotation of tissue-engaging support member 141.

In some embodiments, gear mechanism may be configured so that linear displacement of the support-member steering rod in distal direction D1 may be effected by sliding a slider member, which may embody cutting-actuating handle 131, in the same distal direction D1. Accordingly, linear displacement of cutting-steering rod in proximal direction D2 may be effected by sliding the slider member embodying support-member actuating handle 151 in the same proximal direction D2.

In some other embodiments, gear mechanism may be configured so that linear displacement of cutting-steering rod in distal direction D1 may be effected by sliding a slider member embodying support-member actuating handle 151 in the opposite, proximal, direction D2. Accordingly, linear displacement of cutting-steering rod in proximal direction D2 may be effected by sliding a slider member embodying support-member actuating handle 151 in the reverse, distal direction D1.

Without being construed as limiting and merely to simplify the discussion that follows, displacement of support-member steering rod 146 in distal direction D1 (i.e., towards distal handle portion 111) is in the following considered to cause a clockwise rotation, which is schematically indicated herein by arrow M1.

According to some embodiments, a support-member actuating handle may in some embodiments be implemented differently. A cutting-actuating handle may for instance be implemented as rotating knob 1251, as schematically shown in FIG. 12. Depending on the direction of rotation of such knob 1251, support member steering rod 146 may be displaced in distal direction D1 or proximal direction D2, for respectively rotating tissue-engaging support member 141 in a clockwise or a counterclockwise direction, for example. As already outlined herein, displacement of support-member steering rod 146 in distal direction D1 or proximal direction D2 may in some embodiments cause tissue-engaging support member 141 to respectively rotate in a counterclockwise or a clockwise direction.

Arm member 120 may be operative to receive cutting member 125 at its distal arm end 122. Cutting member 125 may be embodied in various shapes and forms. For example, as illustrated schematically in FIGS. 1A-1D, FIGS. 2-4 and FIG. 6, cutting member 125 may in some embodiments include a cutting blade 126, which may be substantially circular, and that is rotatably mounted at distal arm end 122 such as to rotate or swivel around a second rotating axis Z2 which coincides with the longitudinal axis of arm member 120. Cutting blade 126 may be rotatably mounted to distal arm end 122 by a rotating coupling shaft 127. Cutting blade 126 may taper towards its outer circumference to terminate in a sharp cutting edge 128.

When surgical device 100 is in an operable position, rotation of arm member 120 around first rotational axis Z1 causes cutting blade 126 to traverse a substantially circular path, and optionally to rotate, while cutting an incision in the lens capsule 230.

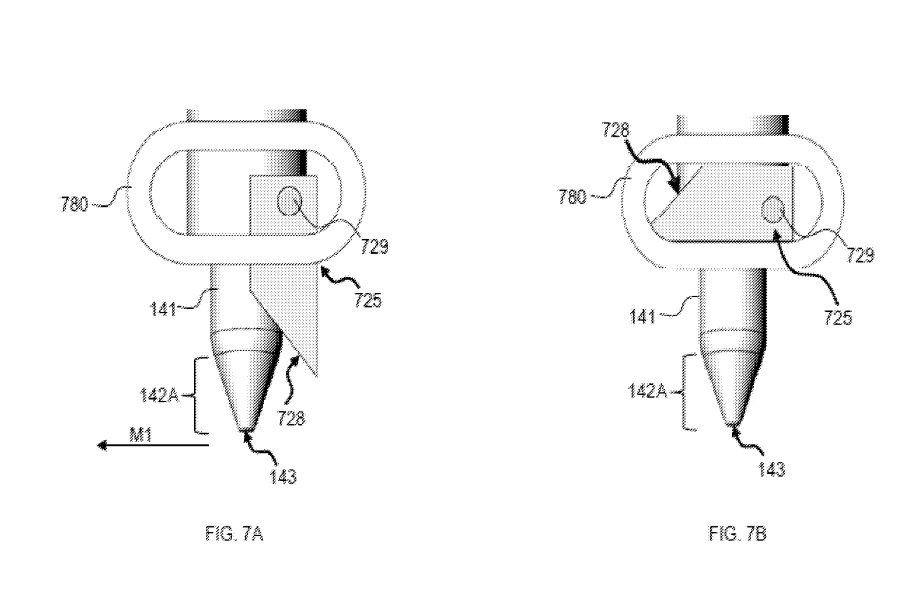

Further reference is made to FIGS. 7A and 7B. In some embodiments, a cutting member 725 may be polygon-shaped, e.g., to implement a scalpel-like function. Cutting member 725 may have a lateral beveled cutting edge 728 extending forward with respect to a rotational cutting direction M1, when surgical device 100 is in an operable position.

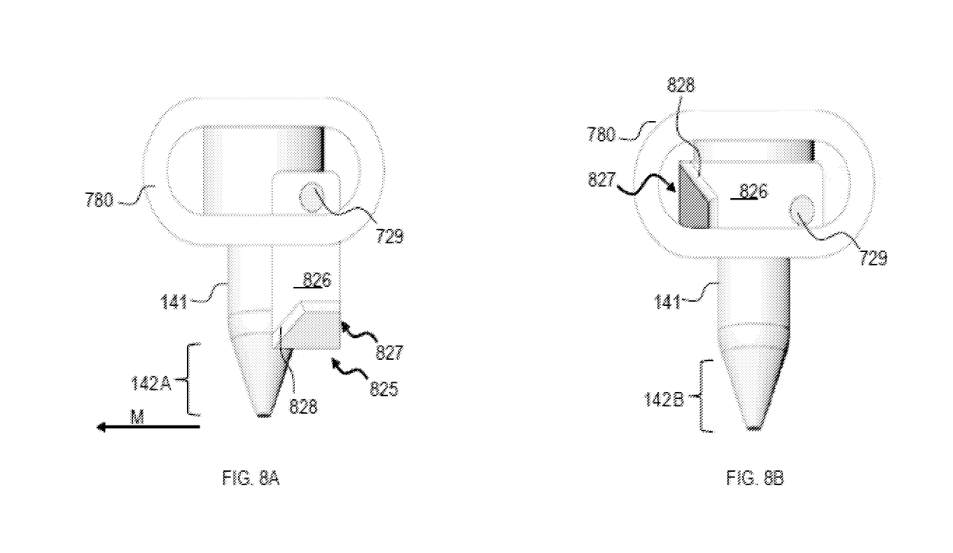

Further referring to FIGS. 8A and 8B, a cutting member 825 may be implemented as or have an L-shaped form. When device 100 is in an operable position, as shown schematically in FIG. 8A, a first leg 826 of the L-shaped form may extend from arm member 120 towards lens capsule 230, and a second leg 827 of the L-shaped form may extend outwardly in a radial direction. Second leg 827 may have a cutting edge 828 facing rotational direction M1. In some embodiments, second leg 827 may be polygon-shaped and/or tapering in a radial direction and/or have any other suitable shape.

As shown schematically in FIGS. 7A and 7B, as well as in FIGS. 8A and 8B, a cutting member may be set (e.g., tilted) from a collapsed or stowed configuration (FIGS. 7A and 8A) into an expanded configuration (FIGS. 7B and 8B), and vice versa. While the possibility of setting a cutting member from a collapsed into an expanded configuration and vice versa is outlined herein with respect to FIGS. 7A to 8B only, this should by no means be construed as limiting. Accordingly, a rotating circular cutting member shown in FIGS. 1A to 1D for instance may also be set from a collapsed or stowed configuration into an expanded position and vice versa.

In some embodiments, a cutting member (e.g., cutting members 125, 725 and/or 825) may be tiltably coupled, e.g., by a tilting pin 729, for allowing the tilting thereof from a collapsed into an expanded position and vice versa.

The cutting member may be set from a collapsed into an expanded position and vice versa by employing a suitable collapsing/expansion arrangement (not shown) comprising an input device allowing the user to manually and one-handedly implement actuation of the collapsing/expansion arrangement.

Surgical device 100 may in some embodiments be equipped with a cut-protection shield 780 encompassing the cutting member when in a collapsed configuration.

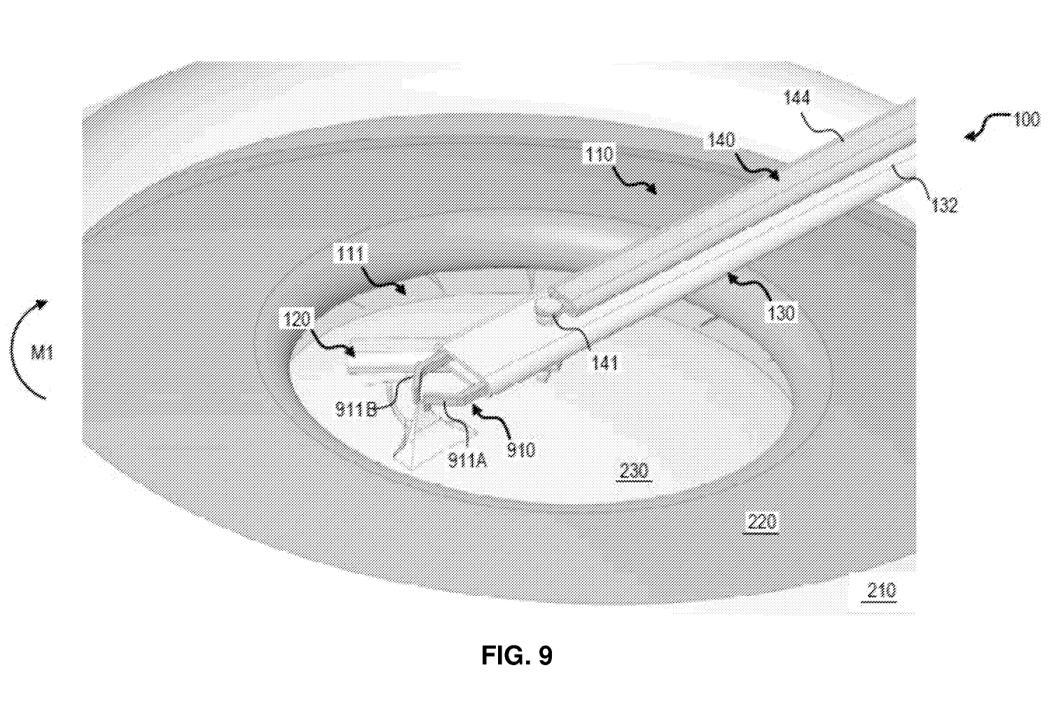

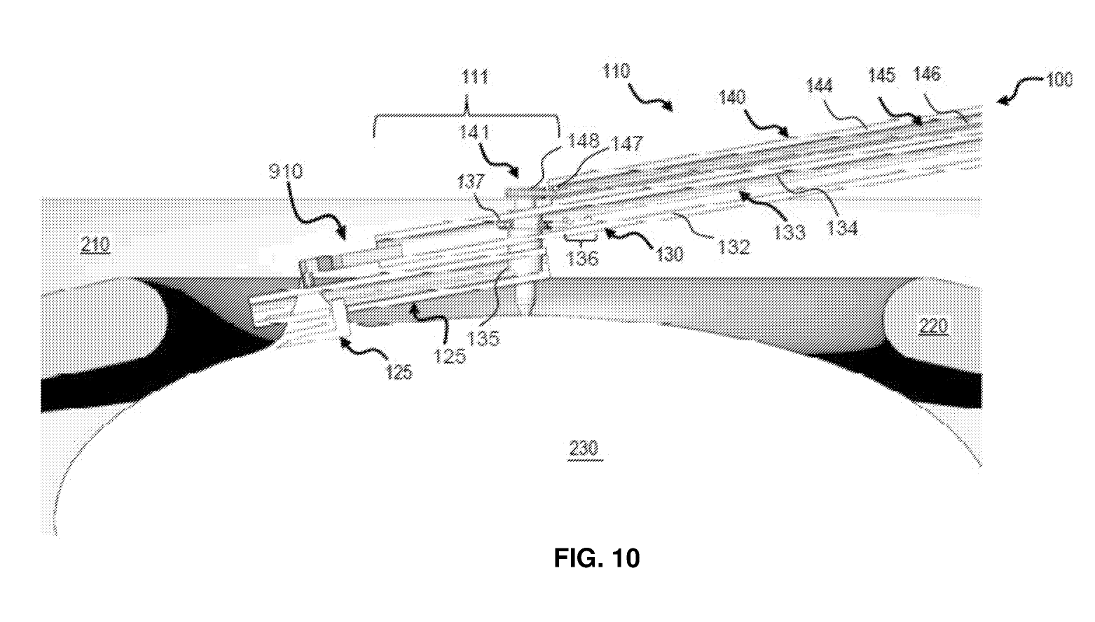

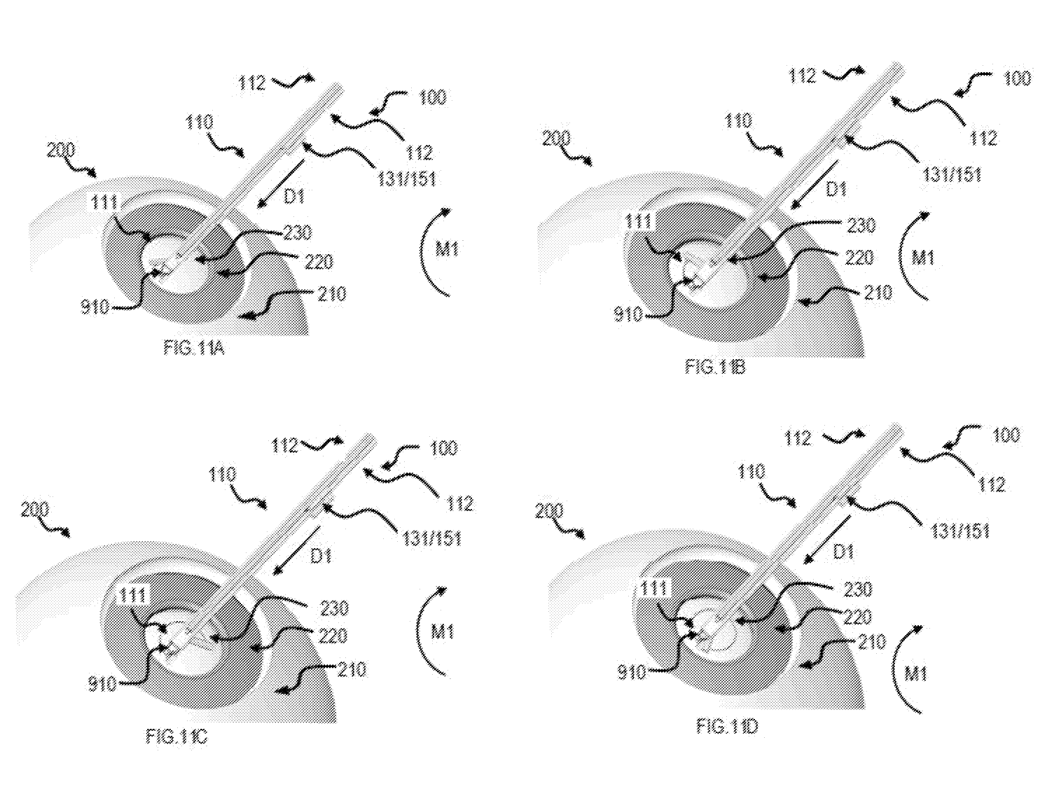

Additional reference is made to FIGS. 9 to 12. According to some embodiments, surgical device 100 may include a tissue-gripper 910. Tissue gripper 910 may be employed for lifting a tissue portion such as, for example, in the membrane tissue of lens capsule 230. By lifting a tissue portion, it may become more stabilized, which, in turn, may increase the counterforce applied by the same tissue portion in response to subjecting it to a cutting force. Such increase in the counterforce may effect a corresponding increase in the forces and/or load within and/or onto the tissue portion, which may facilitate the tearing and/or yielding of the tissue in response to the force applied for cutting the tissue by the cutting member (e.g., cutting member 125, 725 or 825).

Tissue-gripper 910 may comprise a first gripper leg 911A and a second gripper leg 911B, each terminating at a gripper end. These two gripper legs may in some embodiments be joined at a common pivot element (not shown) and lie in a plane that is about in the direction of rotation of arm member 120. In other words, gripper legs 911A and 911B may lie in a plane which is about parallel to a plane that is traversed by rotating arm member 120.

Gripper legs 911A and 911B can be remotely controlled, e.g., manually by the user, for selectively widening or narrowing the distance between the gripper ends of legs 911A and 911B. A gripper leg such as leg 911A and/or 911B may each have three leg portions. First leg portions respective of gripper legs 911A and/or 911B may extend outwardly as an extension of arm member 120. Second leg portions may further extend outwardly yet also inwardly from the first and second leg portions towards each other. Finally, third leg portions extend downwardly from the respective second leg portions. Accordingly, when surgical device 100 is in an operable position, the third leg portions extend towards the lens capsule 230 for grabbing the membrane tissue encapsulating the former.

In an embodiment, at least some or all parts of surgical device 100 may be made of a sterilizable material, e.g., by an autoclave or a suitable gas. Accordingly, in an embodiment, at least one or more elements of surgical device 100 may be configured to allow reuse. In an embodiment, at least some or all parts of surgical device 100 may be made of medical grade material, including, e.g., metal and/or plastic.

In an embodiment, device 100 may be configured to allow for suture-less incision closure. Lateral extensions of surgical device 100 may have dimensions so that an incision length, for example in the cornea near the limbus (not shown), may range, for example, from 0.5 mm to 2.8 mm, 1.8 mm to 2.8 mm, 2 mm to 3 mm, 0.5 mm to 2.5 mm, less than 2.5 mm, less than 2 mm, or any other suitable combination of ranges.

In an embodiment, at least one or more elements of surgical device 100 may be replaceable. For example, cutting member 125 and/or tissue-engaging support member 141 may be disposable and replaceable by another cutting member 125 and/or tissue engaging support member 141, respectively. In some embodiments, all elements may be disposable.

The various features and steps discussed above, as well as other known equivalents for each such feature or step, can be mixed and matched by one of ordinary skill in this art to perform methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

Positional terms such as “upper”, “lower” “right”, “left”, “bottom”, “below”, “lowered”, “low”, “top”, “above”, “elevated”, “high”, “vertical” and “horizontal” as well as grammatical variations thereof as may be used herein do not necessarily indicate that, for example, a “bottom” component is below a “top” component, or that a component that is “below” is indeed “below” another component or that a component that is “above” is indeed “above” another component as such directions, components or both may be flipped, rotated, moved in space, placed in a diagonal orientation or position, placed horizontally or vertically, or similarly modified. Accordingly, it will be appreciated that the terms “bottom”, “below”, “top” and “above” may be used herein for exemplary purposes only, to illustrate the relative positioning or placement of certain components, to indicate a first and a second component or to do both.

The terms “distal” and “proximal” are used herein with respect to a position relative to a user of the surgical device.

It should be understood that where the claims or specification refer to “a” or “an” element, such reference is not to be construed as there being only one of that element.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Unless otherwise stated, the use of the expression “and/or” between the last two members of a list of options for selection indicates that a selection of one or more of the listed options is appropriate and may be made.

All references mentioned in this specification are herein incorporated in their entirety by reference into the specification, to the same extent as if each individual patent was specifically and individually indicated to be incorporated herein by reference. In addition, citation or identification of any reference in this application shall not be construed as an admission that such reference is available as prior art to the present application.

1. A surgical device for assisting a user of the device to cut a circular incision in a tissue, wherein said tissue is an anterior lens capsule tissue, the surgical device comprising:
an arm member, comprising a proximal end and a distal end, wherein the distal end of the arm member is operative to receive a cutting member; and
an actuator arrangement comprising an actuating handle operatively coupled with the arm member, such that, responsive to an operating input provided at the actuating handle, the distal end of the arm member traverses a circular path around a first axis of the tissue to cause the cutting member to form a circular incision in the tissue along the circular path.

2. The surgical device according to claim 1, wherein the actuator arrangement is manually operable with one-hand through the actuating handle.

3. The surgical device according to claim 1, further comprising a tissue-engaging support member extending through a tube-shaped portion of the actuator arrangement, the tissue-engaging support member operative for providing support to the surgical device and fixating the position of the surgical device relative to the tissue.

4. The surgical device according to claim 3, wherein the tissue-engaging support member is rotatable.

5. The surgical device according to claim 4, wherein the tissue-engaging support member comprises form-locking engagement elements.

6. The surgical device according to claim 1, wherein the cutting member comprises a circular blade rotatable around a second axis perpendicular to a first rotational axis of the arm member.

7. The surgical device according to claim 1, wherein the cutting member comprises an L-shaped cutting member.

8. The surgical device according to claim 1, further comprising a handle for allowing the user to hold the device in one hand to allow one-handed operation of the device.

9. The surgical device according to claim 1, comprising a tissue-gripper, operative for gripping and lifting at least a portion of the tissue.

10. A method for cutting a circular incision in a tissue, wherein said tissue is an anterior lens capsule tissue, the method comprising the procedures of:
providing a surgical device comprising:
an arm member, comprising a proximal end and a distal end, wherein the distal end of the arm member is operative to receive a cutting member; and an actuator arrangement comprising an actuating handle operatively coupled with the aim member, and
applying an operating input at the actuating handle, such that the distal end of the arm member traverses a circular path around a first axis of the tissue to cause the cutting member to form a circular incision in the tissue along the circular path.
